(19) United States
(12) Patent Application Publication
(10) Pub. No.: US 2025/0057716 A1
(43) Pub. Date: Feb. 20, 2025

(54) A Portable, Daily Wearable, Smart Compression Device

(71) Applicant: ANATOMECH PVT LTD., Pune (IN)

(72) Inventors: Divyakshi Kaushik, Pune (IN); Aayushree Ghorpade, Nagpur (IN)

(21) Appl. No.: 18/701,283

(22) PCT Filed: Sep. 14, 2022
(86) PCT No.: PCT/IN2022/050822

§ 371 Date: Apr. 15, 2024

(30) Foreign Application Priority Data
Sep. 14, 2021 (IN) 202121041303

(51) Int. Cl. A61H 1/00

(52) U.S. Cl. CPC A61H 1/006; A61H 2201/0111; A61H 2201/0157; A61H 2201/0207; A61H 2201/0214; A61H 2201/0228; A61H 2201/1207; A61H 2201/1638; A61H 2201/1642; A61H 2201/165; A61H 2201/1654; A61H 2201/168; A61H 2201/1697; A61H 2201/5007; A61H 2201/5071; A61H 2201/5082; A61H 2205/065; A61H 2205/10; A61H 2205/12; A61H 2209/00

(57) ABSTRACT

A portable, daily wearable, smart compression device The present invention relates to a compression therapy device (100) for promotion of venous and lymphatic flow and its method of use. Particularly, it relates to a compression therapy device (100) which operates at controlled pressure for management of circulatory and muscular deterioration in the human body due to injury/surgery, strenuous physical activity, age, health, genetics, etc. The compression therapy device (100) includes an innermost layer (101) characterized for contacting with the human skin; a middle layer (102) characterized for enclosing the plurality of technological components; an outer layer (103), characterized for covering, protection, and aesthetic appearance of the device (100), a controller (103), characterized for managing the sequential, continuous or the controlled compression and relaxation of the middle layer components (102) and a fastening means (104) characterized for tightening the device, uniformly placed at different locations.

### FIELD OF THE INVENTION

The present invention relates to a compression therapy device (100) for promotion of venous and lymphatic flow. Particularly, it relates to a compression therapy device (100) with controlled pressure for management of a circulatory and muscular deterioration in the human body due to injury/surgery, strenuous physical activity, age, health, genetics, etc. More particularly, the compression therapy device (100) includes clothing like sleeves, garments, wraps, and/or bandages, socks, exoskins, gloves, stockings, jacket, exoskeletons etc. Even more particularly, it relates to gradual compression therapy for enhancement of venous and arterial blood flow as well as lymphatic drainage from body parts in venous and arterial diseases, lymphodema and other related oedemas.

### BACKGROUND OF THE INVENTION AND DESCRIPTION OF THE PRIOR ART

Compression therapy provides treatment for vascular management and wounds arising from venous insufficiency. Benefits of compression therapy include oedema management and improved venous return. The use of compression therapy is in various clinical indications like treatment and/or prevention of wounds, peripheral vascular disease, leg ulcers, oedema, lymphatic disorders, and other conditions. Compressive pressure can be applied by compression garments, wraps, and/or bandages, collectively referred to as compression wearable devices.

In many conventional compression wearable devices, the amount of compression applied at its interface with an anatomical area is unknown, which leads to suboptimal treatment and detrimental results. For effective management of oedema or swollen part, the accurate pressure is to be applied. Along with accurate measurement of compression pressure, also the clinical need for individual patient varies as per severity of oedema. The amount of compression that wearable compression garment is capable of generating is affected by garment material and construction factor like yarn type and size, elasticity of fabric, fabric structure i.e. layering, stitch pattern, size, and/or density. So prior measurements of actual pressure required and choice of compression device fitting in that pressure range is important.

A conventional compression device fails to provide accurate measurement of actual applied compressive pressure and is expensive. Another disadvantage of such conventional compression devices is that they often have components that are reused from patient to patient, thereby increasing the risk of cross contamination, particularly when utilized in wound care. One more disadvantage of a conventional compression therapy is requirement of use the main power supply (wall outlet), and thus impose total confinement on the patient during treatment. The sleeve of device is big and ungainly, and thus restricts the movement of the limb it encompasses and imposes an aesthetic discomfort.

Reference may be made to WO2014184324 entitled “Decongestion garment for treating lymphodema”, which relates to a compression garment with a multi-layer sheet comprising of inelastic layer and resilient layer. The resilient layer is interposed between the body part and the inelastic layer. The resilient layer comprises of a temperature control means to control the temperature between 36-40 C, heating means, a pressure control means which operates by varying fluid content filled in one or more bladders. The garment has portable energy source. The reference, however, speaks about applying compression pressure of 40 mmHg to a skin interface by the fluid filled in the resilient layer, made of foam or other padding or cushioning materials with densities smaller than or equal to 100 kg/m³.

Reference may be made to U.S. Ser. No. 11/248,591 entitled “Contractible band for use in a wearable garment comprising a shape memory material part”, discloses a textile contractible band for use in a wearable garment for applying pressure to a subject body part for managing the lymphodema or venous return which includes an electrically-contractible shape memory material, in particular a shape memory alloy, or shape memory polymer part, a spring arranged to reverse the contraction of the shape memory material and a textile part comprising of an elastic fabric band and an optical fibre strain sensor.

Reference may be made to U.S. Pat. No. 9,271,890B1 entitled “Compression garment apparatus”, discloses to the a compression garment apparatus for a body part of an human and/or animal having a plurality of attaching means connected to the flexible backing, a plurality of fabric covers, the segmented flex frames having the attached shape memory alloy, A plurality of tensioners, a controller, a sensor and a power supply. The segmented flex frame has spring links and struts that hold the shape memory alloy in X-shaped pattern, a sinusoidal pattern, a modified X-pattern, a plurality of circles pattern, or any various pattern usable with the application. Each segmented flex frame can be adapted to contract the individual spring links when the shape memory alloy contracts and each segmented flex frame can be configured to expand the individual spring links from the contracted shape to an expanded shape when the shape memory alloy relaxes.

Given aforesaid disadvantages in the current therapy available, there is a need for developing a means for easily and accurately determining an actual amount of compression applied at an anatomical area by a compressive pressure device. There is a need for a compression device which is economically constructed. Therefore, there exists the need for a compression device having patient comfort yet having a construction that will aid in providing complete compression to the affected body parts by gradient pressure for maximum drainage of the venous and arterial blood flow and lymphatic fluid. There further exists the need to provide a compression device that is light in weight, portable, ambulant, skin friendly and not bulky.

Therefore, the present invention provides a smart compression device for applying the compressive pressure on a body part, comprising of a multi-layered system, shape memory alloy, a base structure, a plurality of tensioners and a plurality of temperature and a plurality of pressure sensors. The said device exerts sequential, continuous or the controlled compression and relaxation on the oedema at different body parts includes hands, arms, feet, ankles, legs etc.

### OBJECTIVES OF THE INVENTION

The primary objective of present invention is to provide a compression device (100) for promotion of venous and lymphatic flow. It relates to gradual compression therapy for promotion of venous and arterial blood flow as well as lymphatic drainage from body parts in treatment of venous and arterial diseases, lymphodema and other related oedemas.

One more objective of present invention is to stimulate venous and arterial blood flow or drain lymphatic fluid accumulated in various parts of the body by application of controlled pressure compression by a compression device (100). More particularly, controlled pressure applied is in the range of 25-120 mmHg.

One more objective of present invention is to provide a compression device (100) for promotion of venous and lymphatic flow, comprising of a multilayer system wherein, an innermost layer characterized for contacting with human skin, a middle layer encloses plurality of technological components and an outer layer characterized for covering, protection, and aesthetic appearance.

Another objective of present invention is to provide a daily wearable compression device (100) which comprises of woven e-textile based actuator (102a) which is combination of a shape memory alloy (102b) i.e. composite of two or more metal ions, for example Ni, Ti, Cu, Au, He, Fe, Pd and a textile material (102a) like flame resistant and elastic materials with dielectric strength and electrical insulation.

Yet another objective of present invention is an application of the compression device (100) for promoting sequential, unidirectional blood flow from venous and arterial system or drainage of lymphatic system in various parts of body for example, from wrist to shoulder.

Another objective of present invention is to provide a compression device (100) which is portable, ambulant, lightweight, less bulky and skin friendly.

Another objective of present invention is to provide a process for enhancement of venous and arterial blood flow or drainage of lymphatic system by the use of a compression device.

### SUMMARY OF THE INVENTION

The present invention deploys usage of a smart compression device (100) for applying the compressive pressure on body parts particularly hand, arm, feet, ankle and leg, comprising of an innermost layer (101) characterized for contacting with the human skin; a middle layer (102) characterized for enclosing the plurality of technological components; an outer layer (103) characterized for covering, protection, and aesthetic appearance of the device (100), a controller (104) characterized for managing the sequential, continuous or the controlled application of compression and relaxation of the middle layer components (102) and a fastening means (104) uniformly placed at different locations characterized for tightening the device.

According to one aspect of present invention, the innermost layer (101) which comes in contact with the human skin comprises of a lightweight, moisture retaining, highly tensile breathable fabric. The breathable fabric is a quick dry material leads to water transportation with drying. The breathable fabric which is a part of the innermost layer (101) possess weight of 160-170 g/m², moisture regaining capacity of 98-99N/15 mm, and thickness ranging in between 0.5-2.0 mm. The breathable fabric imparts anti-microbial property, anti-fungal property to the skin in contact. The breathable fabric is selected from the group of natural fibers particularly, Bamboo, Hemp, Ramie, Regenerated fibers particularly, Viscose, Lyocell, Modal, Rayon, Synthetic fibers particularly, polyester PES, PET, acrylics etc. More particularly the innermost layer (101) comprises of a breathable fabric, composed with proportions of Nylon Spandex blend fabric.

According to one aspect of the present invention, the middle layer (102) comprises of a base structure (102a) characterized for supporting and disposing the shape memory alloy (102c), augmenting contraction by applying a baseline pressure to the body part and activating a pullback force by acting as a tensioner; a shape memory alloy (102b) characterized for application of contraction; a plurality of anchor points (102c) characterized for attachments of the shape memory alloys (102b); a plurality of temperature sensors (102d) characterized for sensing and controlling temperature of the shape memory alloy (102b); a plurality of pressure sensors (102e) characterized for sensing and controlling pressure of the shape memory alloy (102b), and a resistive material layer (102f) characterized for encasing the technology components and for thermal, electrical and water insulation.

In accordance with one aspect of the present invention, the elastic base structure (102a) acts as a tensioner (102a) for providing springing action. The elastic base structure (102a) applies a force to maintain the required pressure generated by the Shape memory alloy (102b) after actuation due to the power applied from the controller (104) as well as facilitates the pull back or the retaining of the base material (102a) and shape memory alloy (102b) to the original position after turning off the controller (104). The elastic base structure (102a) is situated at each cuff as a base.

In accordance with one aspect of the present invention, the shape memory alloy (102b) is the important technological component present in the middle layer (102), gets actuated after the supply of power by controller (104) and imparts the sequential, continuous or the controlled contraction and relaxation on the affected body parts with oedema. During the one heating phase, after the shape memory alloy (102b) receives current from the controller (104), it raises the temperature of the shape memory alloy (102b) to a target transition temperature between 45° C. to 90° C. and results in shortening of the length of the shape memory alloy (102b). The shape memory alloy (102b) is selected from the composite of two or more metal ions particularly, Ni, Ti, Cu, Au, Hf, Fe, Pd, more particularly Nitinol (Ni). The shape memory alloy (102c) is used in the form of wire, foil, stents, helical springs, flat springs, tubes, mesh. The shape memory alloy (102b) is woven through plurality of anchoring points (102c) and supported on the elastic base structure (102a). The shape memory alloy (102b) is anchored in different arrangements for e.g. waveform arrangement, dual waveform arrangement, quadruple arrangement, intertwined arrangement, paired arrangement. The selection of the arrangement is done based on the circumferential pressure required to be applied. The anchoring points (102c) are holders or struts made of composite materials. Terminals of shape memory alloys (102b) are connected to power source acquainted in the controller (104).

In accordance with one aspect of the present invention, an elastic base structure (102a) serve the purpose of supporting the shape memory alloy (102b) woven through the anchoring points (102c) as well as its also augmenting the contraction generated by the shape memory alloy (102b) all over the affected body part. The elastic material used for base structure possess the tensile strength in the range of 57-59N/15 mm, compression deflection in the range of 50-51 mm, weight in the range of 440-450 g/m2 and the peak load in the range of 57-58N. The constituent of the elastic base structure (102a) is mainly the elastic spandex materials due to its good compressibility property and recovery.

In accordance with one aspect of the present invention, multiple units of the shape memory alloy (102b) in the waveform pattern are woven through the anchoring point (102c) and are connected with each other along the length of the supporting base structure (102a). The plurality of single units of the shape memory alloy (102b) gets actuated in intermittent, sequenced, or continuous manner to assist the compression of the device (100).

In yet another aspect of the invention, the resistive material layer (102f) facilitates encasing of the technology components and provides the thermal, electrical and water insulation. The resistive material layer (102f) is selected from heat and electric arch protecting material, particularly the material used is a Nomex®. Nomex® is specifically designed to meet or exceed the standards required in multi-hazard thermal environments. Different varieties of the Nomex® material used are ASTM F1506, NFPA 2112, CGSB 155.20, ISO 1161, EN 1149, IEC 61482-2, and OEKO-TEX-100.

In yet another aspect of the invention, the middle layer (102) also encloses the highly sensitive temperature (102d) and pressure sensors (102e), sensing and controlling the pressure in the range of 0-150 mmHG and temperature in the range of −20-120° C. respectively.

In one aspect of present invention, the outer layer (103) comprises of breathable fabric with thickness ranging between 0.5-2.5 mm selected from group of blends of Shape Memory Polyurethane (SMPU) with thermal sensitivity particularly, Lycra/Spandex with Nomex/Teflon. The fabric used for the outer layer possesses the weight in the range of 100-200 g/m², tensile strength in the range of 50-100N/15 mm, low stretchability required for stiffness to take the contour of the body part, good moisture absorption and quick drying capacity. The particularly selected fabrics are Looped Fabric or the Nylon Spandex Warp knitted fabric. The main function of the outermost layer is to cover, protect, and provide aesthetic appearance to the device (100).

In yet another aspect of present invention, the PCB unit in controller (104) deports a mechanical movement of the shape memory alloy (102b) through an electrical stimulus. As a result of the electrical stimulus, the plurality of shape memory alloy units (102b) gets actuated and executes the compression action. Once a pressure ranging between 25-125 mmHg and temperature ranging between 45-90° C. is sensed by the temperature and pressure sensors (102d and e), the feedback mechanism is initiated which facilitates the maintenance of the compression between 10-60 sec, and eventually cuts off the electrical stimulus.

In yet another aspect of present invention, the controller (104) applies a current in the range of 0.1-1A, 9-24V for 1-10 sec to the plurality of shape memory alloy strands (102b) at defined intervals to exert intermittent, sequenced, or continuous compression therapy to the body part with the help of the power supply units. The power supply units can be portable batteries. The controller (104) also has a PCB unit, motherboard, microprocessor and other storage device.

In still another aspect of the invention, the compressive pressure applied to the affected area is controlled between 25-125 mmHg as per the requirement of the affected area, in a controlled, dynamic or sequential mode of contraction.

In accordance with one aspect of present invention, the compressible device (100) constituting the multilayer system possess the thickness ranges between 5-15 mm. Out of the multilayer system of the device (100), the thickness of inner layer (101) is ranging between 0.5-2.0 mm and of outer layer (103) it is raging between 0.5-2.5 mm.

In still another aspect of the invention, the instant compressible device (100) can be used for management of lymphatic oedema or lymphatic obstruction caused at the different body parts including hand, arm, feet, ankle and leg. Hence the device (100) is useful for the patients with peripheral oedema, cancer related lymphodema, congestive heart failure, bedridden patients with stagnated fluid flow, abnormal blood pressure, compartment syndrome, restless leg syndrome, diabetic patients, elderly patients, patients with varicose veins, and/or any other person Suffering from peripheral oedema, cramps, or poor circulation.

In still another embodiment of present invention, the device (100) operates as detailed below. The method includes the steps of placing the compression device (100) on the affected body part with the help of the fastening means (105), supplying the power to the plurality of shape memory alloy units (102b) through the controller (104), in case where no current is passing, shape memory alloy (102b) is in a detwinned martensite phase; passing the current of 0.1 A-1A, 9V-24V for 1-10 sec, heats the shape memory alloy (102b) by Joule's heating to 45-90° C. and raising the shape memory alloy (102b) temperature to Austenite final temperature which causes contraction in the Shape memory alloy (102b), which results in contraction of fabric closer to each other and generation of pressure against the circumference of the swollen extremity, cooling of the Shape memory alloy (102b) at −50° C.-50° C. for 20-90 sec. to Martensite final temperature range (Mf) relaxing it back to its original length, releasing the pressure application; and repeating the contraction and the relaxation of Shape memory alloy (102b) after every 60-80 sec.

It has been found according to this invention it is indicative of the fact that the portable, daily wearable, smart compression device (100) is in the form of a compression clothing particularly, suitable for daily wear, light in weight and does not restrict the daily activities.

A further advantageous of the present device and the method of compression device is as it provides lifetime management of venous and arterial diseases, lymphodema and other related oedemas with intermittent, sequenced, or continuous controlled pressure according to swelling condition.

An additional advantage lies in skin compliant layers, easy drying fabric, easily portable and no requirement of a trained person to operate the compression device (100).

### BRIEF DESCRIPTION OF THE ACCOMPANYING DRAWINGS

Reference will be made to embodiments of the invention, examples of which may be illustrated in accompanying figures. These figures are intended to be illustrative, not limiting. Although the invention is generally described in the context of these embodiments, it should be understood that it is not intended to limit the scope of the invention to these particular embodiments.

FIG. 1 represents diagram of a portable, daily wearable, smart compression device (100) and connections between the components

FIG. 2 represents a cross-sectional view showing the layering system of a compression device (100)

FIG. 3 represents a cross-sectional view of the middle layer (102) enclosing the technological components

FIG. 4 represents the single unit of waveform shape memory alloy i.e. Nitinol (102b) woven through the elastic base like structure (102a)

FIG. 5 represents the heating, cooling and standby state of the single unit of the shape memory alloy i.e. Nitinol (102b) woven through elastic base like structure (102a)

FIG. 6 represents graph of the material stroke length vs. the compression the shape memory alloy i.e. Nitinol (102b) after actuation

FIG. 7 represents a process flow for working of a compression device (100)

FIG. 8 represents a perspective view of a person wearing compression sleeves (100)

TABLE No 1
Legend and Legend Description

| Legend | Legend description |
|---|---|
| 101 | An innermost layer which is in contacting with the human |
| | skin; |
| 102 | A middle layer for enclosing the plurality of technological |
| | components; |
| 103 | An outer layer for covering, protection, and aesthetic |
| | appearance of the device (100), |
| 104 | A controller for managing the intermittent, sequenced, or |
| | continuous compression and relaxation of the middle layer |
| | components (102) |
| 105 | A fastening means for tightening the device uniformly placed |
| | at different locations. |
| 102a | A base structure for supporting, disposing the shape memory |
| | alloy (102b), augmentation of contraction, applying pull |
| | back force, and a baseline pressure to the body part; |
| 102b | A shape memory alloy for application of contraction after |
| | actuation; |
| 102c | A plurality of anchor points for attachments of the shape |
| | memory alloys; |
| 102d | A plurality of temperature sensors for sensing and |
| | controlling temperature of the shape memory alloy |
| 102e | A plurality of pressure sensors for sensing and controlling |
| | pressure of the shape memory alloy (102c); |
| 102f | A resistive material layer characterized for encasing the |
| | technology components and for thermal, electrical and water |
| | insulation |

### DETAILED DESCRIPTION OF THE INVENTION

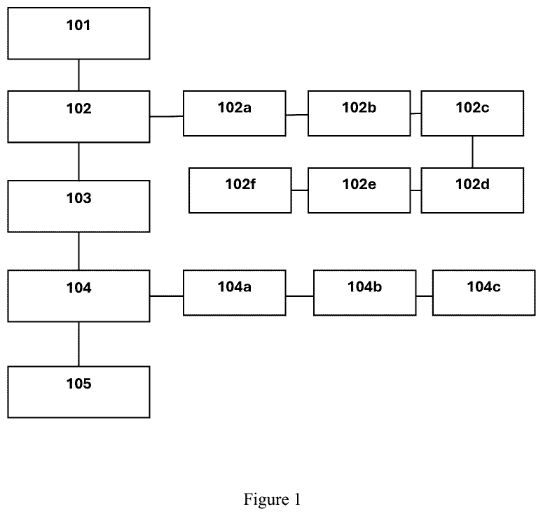

According to one embodiment of present invention, FIG. 1 represents diagram of the portable, daily wearable, smart compression device (100) and connections between the components according to an embodiment of the present invention. The device (100) comprises of an innermost layer (101) which is in contact with the skin; a middle layer (102) characterized for enclosing the plurality of technological components; an outer layer (103), characterized for covering, protection, and aesthetic appearance of the device (100), a controller (104), characterized for managing the sequential, continuous or the controlled compression and relaxation of the middle layer components (102), and a fastening means (105) characterized for tightening the device, uniformly placed at different locations. The innermost layer (101) comprises of breathable fabric with high moisture regain, anti-microbial property, anti-fungal property, tensile strength, and thickness ranging in between 0.5-2.0 mm, selected from the group of natural fibers particularly, Bamboo, Hemp, Ramie, Regenerated fibers particularly, Viscose, Lyocell, Modal, Rayon, Synthetic fibers particularly, polyester PES, PET, acrylic. The middle layer (102) comprises of a plurality of tensioners (102a) backed on a spandex fabric, characterized for applying pull back force, and a baseline pressure to the body part; an elastic base structure mainly a spandex elastic fabric (102a), characterized for supporting, disposing the shape memory alloy (102b), augmentation of contraction, applying pull back force, and a baseline pressure to the body part; a shape memory alloy (102b), characterized for application of contraction; a plurality of anchor points (102c), characterized for attachments of the shape memory alloys; a plurality of temperature sensors (102d), characterized for characterized for sensing and controlling temperature of the shape memory alloy (102b); a plurality of pressure sensors (102e), characterized for sensing and controlling pressure of the shape memory alloy (102b); and a resistive material layer (102f), characterized for encasing the technology components and for thermal, electrical and water insulation. The outer layer (103) comprises of breathable fabric selected from group of blends of Shape Memory Polyurethane (SMPU) with thermal sensitivity particularly, Lycra/Spandex with Nomex/Teflon with thickness ranging between 0.5-2.5 mm. The controller (104) is mounted outside of the layered system of a compression device (100) and manages the intermittent, sequenced, or continuous compression and relaxation of plurality of shape memory alloy units (102c) woven through the elastic base structure (102a) with the help of anchoring points (102c). The Controller (104) comprises of battery (104a), mother board (104b) and other electronic components (104c) that assist in sequential, continuous or the controlled compression and relaxation of the plurality of shape memory alloy units (102b).

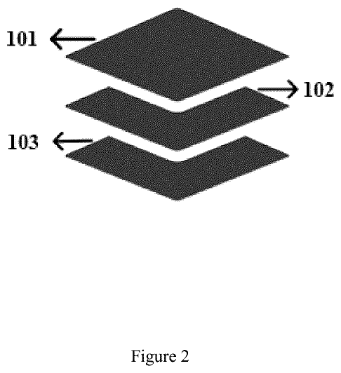

According to another embodiment of present invention, FIG. 2 represents a cross-sectional view of the multi-layering system of a compression device (100). The compression device (100) is a multi-layered system with the innermost layer (101), the middle layer (102) and outer layer (103). The three key layers are stacked over one another. The inner layer (101) is in contact with the skin, hence the inner layer (101) possess the skin-friendly, moisture management and quick dry property and comprises mainly nylon, poly, modal, viscose, spandex blend. The middle layer mainly comprises of the technological components (102a-g) enclosed for sequential, continuous or the controlled application of pressure on the affected area. The outer layer (103) helps in moisture and heat translation to atmosphere as well as body temperature control.

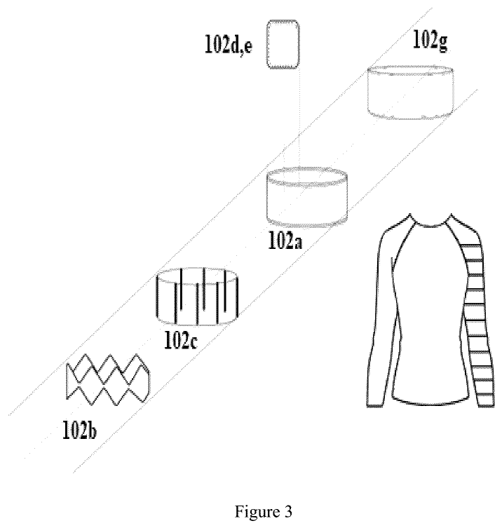

According another embodiment of present invention, FIG. 3 represents a cross-sectional view of the middle layer (102) enclosing the technological components. The technological components comprises of an elastic base structure (102a) mainly a spandex fabric used as a tensioner, assist in applying the desired pressure for compression, maintaining the pressure, the applying the pullback force required for relaxation of pressure applied to the compression device (100) and supporting the plurality of shape memory alloy (102b). The technological component also further comprises of the multiple units of shape memory alloy (102b) particularly Nitinol woven through the plurality of anchoring points (102c) over the base structure (102a), arranged in the waveform pattern which contracts or deforms after passing the current 0.1A-1A, 9V-24V for 1-10 sec due to heating up of the shape memory alloy (102b) particularly Ni to Austenite temperature that results in its deformation. The technological layer also has the temperature and pressure sensors (102d and e) for sensing, monitoring and controlling the temperature of the multiple units of shape memory alloy (102b) and the compressible pressure applied through the device (100). The plurality of temperature and pressure sensors (102d and e) are placed over the plurality of shape memory alloy units (102b) arrange in waveform at the intersection of two units. The technological layer further has a PCB unit in controller (104) for accepting the controls from the temperature and pressure sensors (102d and e), controller (104) and it passes the signal to the controller and the technological components for application of compression. All the technological components are encased in the resistive material layer (102f) for thermal, electrical and water insulation.

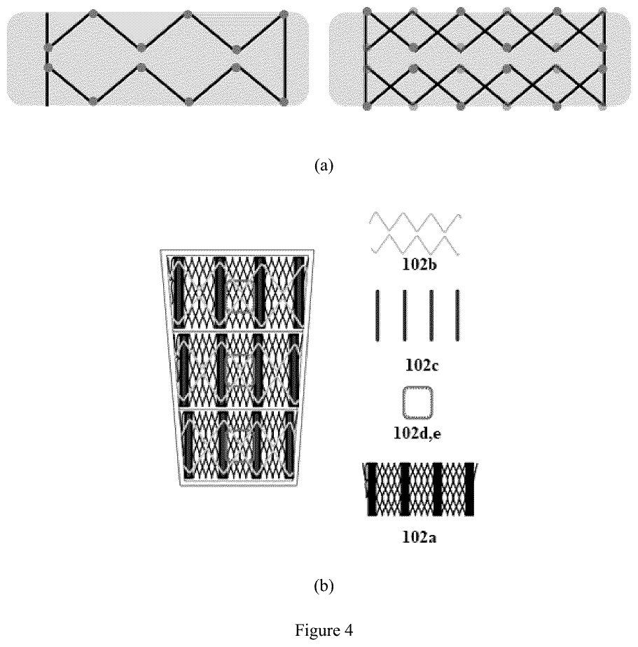

According to another embodiment of present invention, FIG. 4a represents the single unit of waveform shape memory alloy i.e. Nitinol (102b). The FIG. 4a depicts the waveform shape memory alloy (102b) particularly Nitinol in the wired form of tied up at the anchoring points (102c) over the collapsible elastic base material (102a). The anchoring points (102c) include holders or struts which are made up of composite materials and equidistantly place in a waveform pattern. The arrangement involves intersection of the two wires of waveform shape memory alloy (102b). At the point of intersection the temperature and pressure sensors (102 d and e) are situated, as represented in FIG. 4b. So according another embodiment of present invention FIG. 4b represents the multiple units of waveform shape memory alloy i.e. Nitinol (102b) are woven through elastic base structure (102a) with the help of anchoring points (102c) the multiple units of waveform shape memory alloy i.e. Nitinol (102c) and encased in the protective encasing.

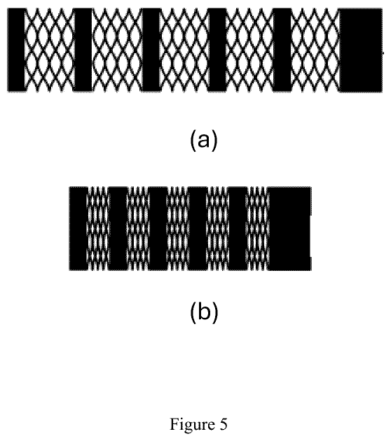

Yet another embodiment of present invention represents, FIG. 5 for standby state and the heating state of the single unit of the shape memory alloy i.e. Nitinol (102b) woven through elastic base structure (102b). The standby phase herein refers to the interval of time when the temperature of the single unit of shape memory alloy, particularly Nitinol (Ni) (102b) is held just below a target transition temperature, such as −50-50° C. In the standby phase, a length of the shape memory alloy (102b) remains unchanged. In the standby phase, at the room temperature, the shape memory alloy (102b) leads to the lengthening of shape memory alloy (102b). As a result of the standby phase of the shape memory alloy (102b), the collapsible elastic the base structure (102a) remains in the relaxed state, as depicted in FIG. 5a. The heating phase of the unit of the shape memory alloy, particularly Nitinol (Ni) (102b) refers to a time period during which a length of the shape memory alloy is warmed to reach a target transition temperature in the range of 45-90° C. to shorten the shape memory alloy (102b) by 3% to 5%. As the result of the shortening of the single unit of shape memory alloy (102b), the elastic base structure (102a) anchored with the shape memory alloy (102b) tends to remain in contracted state and applies the compressible pressure, as depicted in FIG. 5b.

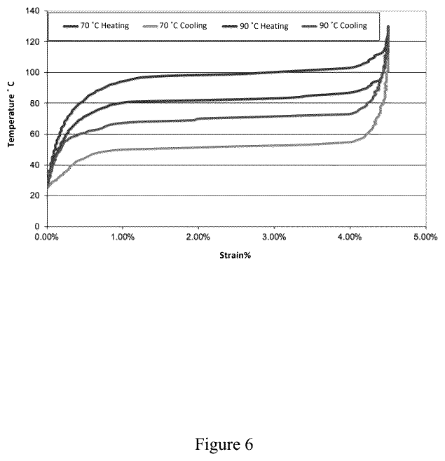

According another embodiment of the present invention, FIG. 6 represents the graph of the material stroke length vs. the compression the shape memory alloys i.e. Nitinol (102b) after actuation. Typical Temperature vs. Strain Characteristics for Dynalloy's standard 158° F. (70° C.) “LT” and 194° F. (90° C.) “HT” Austenite start temperature alloys, at 172 MPa.

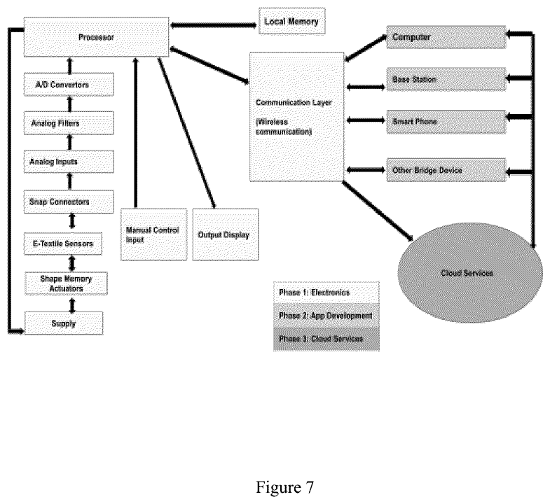

According to one of the embodiment of present invention, FIG. 7 represents a process flow for working of the compression device (100), which includes an application of the compression device (100) which includes sleeves, garments, wraps, and/or bandages, socks, exoskins, gloves, stockings, jacket, exoskeletons on the affected body part and adjusting the required the pressure to be applied through the controller (104) on the swollen area, after pressure adjustment, inside the compression device (100) the multiple units of the shape memory alloy (102b) gets actuated. In case where no current is passing, the plurality of units of the shape memory alloy (102b) are in a detwinned martensite phase, after passing the current of 0.1-1 A, 9V-24V for 1-10 sec, it heats the shape memory alloy (102b) by Joule's heating to 45-90° C. and resulting in rise in temperature to Austenite final temperature i.e. −130° C. to 220° C. Heating of Shape memory alloy (102b) causes contraction in the Shape memory alloy (102b), which results in contraction of elastic base material (102a) and generation of pressure against the circumference of the swollen extremity. The cooling of the Shape memory alloy (102b) at −50-50° C. for 20-90 sec. to Martensite i.e. Martensite start temperature range (Ms) −190° C. to 160° C., Martensite final temperature range (Mf) −170° C. to 180° C., results in relaxing the plurality of units of the shape memory alloy (102b) and the elastic base structure (102a) back to its original length, releasing the pressure applied. The modes on which the compressible device (100) works involve repetition contraction and relaxation cycles sequentially or continuously or in the dynamic mode according to the need of the affected area.

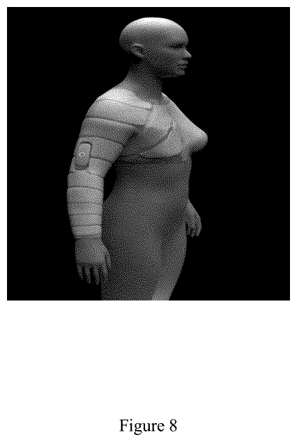

According one aspect of present invention, FIG. 8 represents a perspective view of a person wearing compression sleeves.

### EXAMPLES

The following examples are given by way of illustration only and therefore should not be construed to limit the scope of the present invention in any manner.

Example 1: Selection of the innermost layer (101)—The material from the innermost layer (101) is selected from the group of natural fibers particularly, Bamboo, Hemp, Ramie, Regenerated fibers particularly, Viscose, Lyocell, Modal, Rayon, Synthetic fibers particularly, polyester PES, PET, acrylic. We tested the tensile strength of the materials to be used in innermost layer (101) by using the Universal Testing Machine (UTM), moisture absorbing capacity by the Drop Test and weight assessment by the using weighing machine.

Example 2: Selection of the material for collapsible elastic base structure (102a)—Depending on tensile strength: We tested the materials used for the collapsible elastic base structure (102a) by the routine protocol for testing the tensile strength by using the Universal Testing Machine (UTM). The material of choice for the collapsible elastic base structure (102a) is tested for the Push back capacity i.e. retaining capacity of the material. The Push back capacity is evaluated by the spring Testing. The protocol followed is loading the spring by a suitable weight and noting the corresponding axial deflection in compression, increasing the load and taking the corresponding axial deflection readings. The graph is plotted between load and deflection. The shape of the curve provides the stiffness of the spring. The compressibility of the collapsible elastic base structure (102a) is tested by the Flexure/Bend Testing. The protocol followed is supporting the specimen on a support span and the load is applied to the centre by the loading nose producing three points bending at a specified rate. The parameters for this test are the support span, the speed of the loading, and the maximum deflection for the test.

Example 3: Selection of the material for outermost layer (103)—The material from the innermost layer (103) is selected from group of blends of Shape Memory Polyurethane (SMPU) with thermal sensitivity particularly, Lycra/Spandex with Nomex/Teflon etc. We tested the tensile strength of the different materials by using the Universal Testing Machine (UTM), moisture absorbing capacity by the Drop Test and weight assessment by the using weighing machine.

Example 4: Selection of Shape memory alloy (102b): The material for the shape memory alloy (102c) is tested for the movement test, resistance test by using Digital multimeter against a fixed length, cooling deformation force.

TABLE 2
Comprehensive results for the particularly preferred material
for the innermost layer (101), outermost layer (103),
and collapsible mesh elastic base structure (102b)

| | Innermost layer | Outermost layer | Elastic base |
|---|---|---|---|
| | (101) | (103) | structure (102a) |
| Preferred material | Nylon Spandex | Nylon Spandex | EVA foam |
| | blend fabric | Warp knitted | |
| | | fabric | |
| Tensile strength | 98.01N/15 mm | 66.67N/15 mm | 57.28N/15 mm |
| Moisture | 2 min | Water passes | Zero |
| absorbing | | through | |
| capacity | | | |
| Weight | 166 g/m2 | 149 g/m2 | 446 g/m2 |

### Advantages of the Invention

1. Easy portability of a compression device (100), more particularly a compression clothing
2. Suitable for daily wearing of a compression device (100)
3. Lifetime management of venous and arterial diseases, lymphodema and other related oedemas
4. Pressure control or application of desired pressure according to swelling condition is possible
5. No requirement of skilled person for application and monitoring of a compression device (100)
6. Lightweight of a compression device (100)
7. Skin friendly layers of a compression device (100)
8. No restrictions on daily activities after wearing a compression device (100)

1. A smart compression device (100) for applying the compressive pressure on body parts, comprising:
a. An innermost layer (101), characterized for contacting with the human skin;
b. A middle layer (102), characterized for enclosing the plurality of technological components;
c. An outer layer (103), characterized for covering, protection, and aesthetic appearance of the device (100);
d. A controller (104), characterized for managing the intermittent, sequenced, or continuous compression and relaxation of the middle layer components (102), and
e. A fastening means (105), characterized for tightening the device, uniformly placed at different locations.

2. The smart compression device (100) for applying the compressive pressure on a body parts as claimed in claim 1, wherein the middle layer (102) comprising;
a. An elastic base structure (102a), characterized for supporting and disposing the shape memory alloy (102b), augmenting the contraction, acting as a tensioner, applying pull back force, and a baseline pressure to the body part;
b. A shape memory alloy (102b), characterized for application of contraction;
c. A plurality of anchor points (102c), characterized for attachments of the shape memory alloys (102b);
d. A plurality of temperature sensors (102d), characterized for characterized for sensing and controlling temperature of the shape memory alloy (102b);
e. A plurality of pressure sensors (102e), characterized for sensing and controlling pressure of the shape memory alloy (102b), and
f. A resistive material layer (102f), characterized for encasing the technology components and for thermal, electrical and water insulation.

3. The smart compression device (100) for applying the compressive pressure on body parts as claimed in claim 1, wherein the innermost layer (101) comprises of breathable fabric with high moisture regain, anti-microbial property, anti-fungal property, tensile strength and thickness ranging in between 0.5-2.0 mm, selected from the group of natural fibers particularly, Bamboo, Hemp, Ramie, Regenerated fibers particularly, Viscose, Lyocell, Modal, Rayon, Synthetic fibers particularly, polyester PES, PET, acrylic.

4. The smart compression device (100) for applying the compressive pressure on a body parts as claimed in claim 1, wherein outer layer (103) comprises of breathable fabric with thickness ranging between 0.5-2.5 mm, selected from group of blends of Shape Memory Polyurethane (SMPU) with thermal sensitivity particularly, Lycra/Spandex with Nomex/Teflon.

5. The smart compression device (100) for applying the compressive pressure on a body parts as claimed in claim 1, wherein the shape memory alloy (102b) is in the form of wire, foil, stents, helical springs, flat springs, tubes, mesh particularly anchored in waveform, dual waveform or Quadrupole Waveform arrangement.

6. The smart compression device (100) for applying the compressive pressure on a body parts as claimed in claim 1, wherein the shape memory alloy (102b) is in waveform arrangement, selected from a composite of two or more metal ions particularly, Ni, Ti, Cu, Au, Hf, Fe, Pd with operating temperatures between −I 90° C. to 220° C., more particularly Nitinol.

7. A method for applying the compressive pressure on a body, comprising steps of
a. Placing the compression device (100) on the affected body part with the help of the fastening means (105);
b. Supplying the power to the plurality of shape memory alloy units (102b) through the controller (104);
c. In case where no current is passing, shape memory alloy (102b) is in a detwinned martensite phase;
d. Passing the current of 0.IA-IA, 9V-24V for 1-IO sec, heats the shape memory alloy (102b) by Joule's heating to 45-90° C. and raising the shape memory alloy (102b) temperature to Austenite final temperature which causes contraction in the Shape memory alloy (102b), which results in contraction of fabric closer to each other and generation of pressure against the circumference of the swollen extremity;
e. Cooling of the Shape memory alloy (102b) at −50° C.-50° C. for 20-90 sec. to Martensite final temperature range (Mf) relaxing it back to its original length, releasing the pressure application, and
f. Repeating the contraction and the relaxation of Shape memory alloy (102b) after every 60-80 sec.

8. The method for applying the compressive pressure on a body parts as claimed in claim 5, wherein the pressure applied throughout the swollen area is adjusted in the range of 25-120 mmHg.

9. The method for applying the compressive pressure on a body parts as claimed in claim 5 wherein the compression device (100) is placed on the affected body parts, particularly hand, arm, feet, ankle and leg swollen due to oedema.

10. A smart compression device (100) for treatment of venous and arterial diseases, lymphedema and other related edemas by applying the compressive pressure on body parts, comprising:
a. An innermost layer (101), characterized for contacting with the human skin;
b. A middle layer (102), characterized for enclosing the plurality of technological components;
c. An outer layer (103), characterized for covering, protection, and aesthetic appearance of the device (100);
d. A controller (104), characterized for managing the intermittent, sequenced, or continuous compression and relaxation of the middle layer components (102), and
e. A fastening means (105), characterized for tightening the device, uniformly placed at different locations.

11. The smart compression device (100) for treatment of venous and arterial diseases, lymphedema and other related edemas by applying the compressive pressure on a body parts as claimed in claim 1, wherein the middle layer (102) comprising;
a. An elastic base structure (102a), characterized for supporting and disposing the shape memory alloy (102b), augmenting the contraction, acting as a tensioner, applying pull back force, and a baseline pressure to the body part;
b. A shape memory alloy (102b), characterized for application of contraction;
c. A plurality of anchor points (102c), characterized for attachments of the shape memory alloys (102b);
d. A plurality of temperature sensors (102d), characterized for characterized for sensing and controlling temperature of the shape memory alloy (102b);
e. A plurality of pressure sensors (102e), characterized for sensing and controlling pressure of the shape memory alloy (102b), and
f. A resistive material layer (102f), characterized for encasing the technology components and for thermal, electrical and water insulation.

12. The smart compression device (100) for treatment of venous and arterial diseases, lymphedema and other related edemas by applying the compressive pressure on body parts as claimed in claim 1, wherein the innermost layer (101) comprises of breathable fabric with high moisture regain, anti-microbial property, anti-fungal property, tensile strength and thickness ranging in between 0.5-2.0 mm, selected from the group of natural fibers particularly, Bamboo, Hemp, Ramie, Regenerated fibers particularly, Viscose, Lyocell, Modal, Rayon, Synthetic fibers particularly, polyester PES, PET, acrylic.

13. The smart compression device (100) for treatment of venous and arterial diseases, lymphedema and other related edemas by applying the compressive pressure on a body parts as claimed in claim 1, wherein outer layer (103) comprises of breathable fabric with thickness ranging between 0.5-2.5 mm, selected from group of blends of Shape Memory Polyurethane (SMPU) with thermal sensitivity particularly, Lycra/Spandex with Nomex/Teflon.

14. The smart compression device (100) for treatment of venous and arterial diseases, lymphedema and other related edemas by applying the compressive pressure on a body parts as claimed in claim 1, wherein the shape memory alloy (102b) is in the form of wire, foil, stents, helical springs, flat springs, tubes, mesh particularly anchored in waveform, dual waveform or Quadrupole Waveform arrangement.

15. The smart compression device (100) for treatment of venous and arterial diseases, lymphedema and other related edemas by applying the compressive pressure on a body parts as claimed in claim 1, wherein the shape memory alloy (102b) is in waveform arrangement, selected from a composite of two or more metal ions particularly, Ni, Ti, Cu, Au, Hf, Fe, Pd with operating temperatures between −I 90° C. to 220° C., more particularly Nitinol.

16. (canceled)

17. The method for treatment of venous and arterial diseases, lymphedema and other related edemas by applying the compressive pressure on a body parts as claimed in claim 5, wherein the pressure applied throughout the swollen area is adjusted in the range of 25-120 mmHg.

18. The method for treatment of venous and arterial diseases, lymphedema and other related edemas by applying the compressive pressure on a body parts as claimed in claim 5 wherein the compression device (100) is placed on the affected body parts, particularly hand, arm, feet, ankle and leg swollen due to oedema.
